(19) United States
(12) Patent Application Publication
(10) Pub. No.: US 2026/0137888 A1
(43) Pub. Date: May 21, 2026

(54) Airway Obturator and Method of Intubation Using the said Obturator with a Video Bougie or an Optical Stylet

(71) Applicants: Alan Henry SHIKANI, Towson, MD (US); Acera LLC, Beverly, MA (US)

(72) Inventor: Alan Henry SHIKANI, Towson, MD (US)

(21) Appl. No.: 19/451,339

(22) Filed: Jan. 16, 2026

Related U.S. Application Data
(63) Continuation-in-part of application No. 19/307,675, filed on Aug. 22, 2025.
(60) Provisional application No. 63/686,011, filed on Aug. 22, 2024.
(60) Provisional application No. 63/833,776, filed on Jan. 16, 2025.
(60) Provisional application No. 63/782,327, filed on Apr. 2, 2025.

(51) Int. Cl. A61M 16/04; A61B 1/00; A61B 1/05; A61B 1/06; A61B 1/267; A61M 25/00; A61M 25/01; G09B 5/06

(52) U.S. Cl. CPC A61M 16/0488; A61B 1/05; A61B 1/0615; A61B 1/063; A61B 1/0684; A61B 1/267; A61M 16/0463; G09B 5/065; A61B 1/00016; A61M 2025/0047; A61M 2025/006; A61M 25/0102; A61M 2202/0208; A61M 2205/583

(57) ABSTRACT

In one aspect, a kit for facilitating airway intubation is disclosed, which includes a video bougie comprising an elongated body and a camera system disposed at a distal end thereof, the camera system configured to generate video images of an anatomical lumen, and an oropharyngeal airway obturator comprising a body being configured to slidably receive said video bougie to facilitate insertion of an airway tube into a patient.

### RELATED APPLICATIONS

The present application claims priority to Provisional Patent Application No. 63/833,776, filed on Jan. 16, 2025. Further the present application claims priority as a continuation-in-part application to U.S. patent application Ser. No. 19/307,675, filed on Aug. 22, 2025, which in turn claims priority to Provisional Application No. 63/686,011 filed on Aug. 22, 2024, and Provisional Application No. 63/782,327 filed on Apr. 2, 2025. All of these applications are herein incorporated by reference in their entireties.

### TECHNICAL FIELD

The present disclosure relates generally to upper airway/endotracheal intubation systems and methods for performing intubation.

### BACKGROUND

Endotracheal intubation is a common and often life-saving medical procedure used to establish a secure airway for ventilation. The procedure is traditionally performed using a laryngoscope to visualize the larynx and guide an endotracheal tube (ETT) through the vocal cords. However, this process is frequently complicated by the presence of a “difficult airway,” where direct visualization is obstructed. Various anatomical and physical factors can be associated with difficult airway intubation, including a short, thick neck, limited neck extension or poor range of motion of the cervical spine (such as due to conditions like ankylosing spondylitis or rheumatoid arthritis), small mandible (micrognathia) or a receded jaw (retrognathia), restricted jaw range of motion (trismus) or a small mouth opening (inter-incisor gap less than 3-4 cm); oral and pharyngeal structures abnormalities including large tongue (macroglossia), prominent upper incisors or a significant overbite, high-arched or narrow palate, high Mallampati score (Class 3 or 4), where the uvula or soft palate is not fully visible; acquired diseases and conditions including metabolic and endocrine pathologies such as obesity, especially morbid obesity, obstructive sleep apnea (OSA), endocrine diseases such as thyroid disorders (e.g., goiter causing tracheal deviation/stenosis) and long-term diabetes mellitus, acromegaly (overproduction of growth hormone), infections and trauma, including airway infections like Ludwig's angina or epiglottitis, head, neck, or facial trauma; presence of blood, vomit, or secretions in the airway, intraoral or neck masses, tumors, cancers, abscesses, or hematomas along with various other conditions, including post-radiation changes or surgical scarring in the neck, stiff lungs or cervical spine issues; congenital syndromes including several genetic conditions associated with anatomical variations that make intubation challenging, such as Down syndrome, Pierre Robin syndrome, Treacher Collins syndrome, and Goldenhar syndrome.

To improve access and manage the airway, clinicians often employ aids such as oropharyngeal airway obturators. These devices are designed to be inserted into the mouth to depress and retract the tongue, preventing it from falling back and obstructing the airway path, thereby creating a clearer passage toward the larynx.

However, while such airway devices are effective at managing the tongue, the critical task of accurately guiding the ETT into the larynx remains a significant challenge. Even with a clearer passage, the operator must still navigate the ETT assembly past the epiglottis and into the correct opening, a step that is prone to error and that requires special training and expertise. To address this guidance challenge, practitioners often separately employ an endotracheal tube introducer, commonly known as a “bougie.” A bougie is a thin, semi-rigid guide that is first passed into the trachea, over which the ETT is then slid. The primary drawback of this technique is that the bougie is conventionally inserted “blindly.” forcing the practitioner to rely on unreliable tactile feedback like “tracheal clicks” or a “hold-up” to confirm placement. This blind insertion carries a significant risk of misplacement into the esophagus or, due to the bougie's stiffness, injury to the larynx, and/or perforation of the trachea or bronchi.

### SUMMARY

In one aspect, a kit for facilitating airway intubation is disclosed, which includes a video bougie comprising an elongated body and a camera system disposed at a distal end thereof, where the camera system is configured to generate video images of an anatomical lumen. The kit further includes an oropharyngeal airway obturator comprising a body being configured to slidably receive said video bougie to facilitate insertion of an endotracheal tube into a patient's airway.

In various embodiments, the obturator includes a guiding channel, e.g., a central guiding channel, that extends along at least a portion of its length and is dimensioned to slidably receive the video bougie. By way of example, the guiding channel can be in the form of a groove.

In various embodiments, the obturator includes a generally U-shaped body that is defined by two upwardly extending side portions between which said guiding channel is formed. In some such embodiments, the guiding channel is formed along a central portion of the U-shaped body.

In various embodiments, the obturator further includes at least one proximate bite-block that is configured to prevent dislodgement of the obturator from a patient's mouth and a proximal flange that is configured to prevent over-insertion of the obturator into a patient's mouth.

In various embodiments, the obturator body further includes at least one ancillary conduit that is configured for at least one of: an application of a suction or delivery of oxygen to the patient. In some such embodiments, the at least one ancillary conduit includes a pair of conduits, where one of the conduits is configured for application of the suction and the other conduit is configured for delivery of the oxygen.

In various embodiments, the obturator body further includes a reference marker that is printed along the central axis of the U-shaped channel thereof to provide a visual cue to an operator to guide a bougie-tube or a stylet-tube assembly and facilitate its insertion straight to the middle of the glottis.

In various embodiments, the video bougie further includes a red light source configured to provide transillumination through a patient's tissue. In some such embodiments, the at least one red light source includes a plurality of side-mounted light emitting diodes (LEDs) configured to emit light through one or more windows in a wall of the video bougie.

In various embodiments, the kit can further include an endotracheal tube (ETT) that is configured to be pre-loaded onto the video bougie to form an intubation assembly.

In various embodiments, the obturator is formed of a polymeric material and the guiding channel has a top low-friction surface to facilitate sliding the video bougie along that surface.

In a related aspect, an oropharyngeal airway obturator is disclosed, which includes a body extending from a proximal end to a distal end, the body having an anatomically curved glossal surface and a pharyngeal surface, and a guiding channel (e.g., a central guiding channel) formed along the pharyngeal surface, the channel being open along its length and dimensioned to slidably receive an intubating assembly; and at least one bite-block and a flange disposed at the proximal end of the body.

In various embodiments, the body has a generally U-shaped cross-section defining the central guiding channel. In some such embodiments, a visual midline marker can be printed on a top surface of the central guiding channel.

In various embodiments, the body further includes at least two ancillary conduits extending from the proximal end toward the distal end, a first conduit for suction/removal of saliva, secretions or blood from the airway and a second conduit for oxygen delivery.

The obturator can be formed, e.g., of a variety of polymeric materials with the guiding channel having a low-friction surface.

In a related aspect, a method for intubating a patient is disclosed, which includes inserting an oropharyngeal airway obturator into a patient's mouth to retract the patient's tongue, the obturator having an open guiding channel; placing an intubation assembly into the guiding channel of the inserted obturator, the intubation assembly comprising a video bougie or an optical stylet and an endotracheal tube (ETT); advancing the intubation assembly along the guiding channel toward the patient's larynx while viewing real-time video images generated by a camera on the video bougie or by the optical stylet; and guiding the airway tube into the patient's trachea under visual guidance from the video images with direct visualization of the midline marker and the airway glottis.

In various embodiments, the method further includes withdrawing the video bougie or the optical stylet from the ETT, and removing the oropharyngeal airway obturator from the patient's mouth.

In various embodiments, the method further includes activating a red light source on the video bougie or the optical stylet to generate transillumination, and externally observing the transillumination to confirm a position of the video bougie or the optical stylet to facilitate said step of guiding the airway tube just beyond the glottis and not too deep into the patient's trachea to avoid any injury to the carina or the distal bronchi, and to avoid any unilateral intubation of one lobe of the lung.

In various embodiments, the method further includes the step of administering suction or delivering oxygen through at least one ancillary conduit in the oropharyngeal airway obturator during induction and prior to as well as throughout the intubation.

In a related aspect, an intubation system is disclosed, which includes an oropharyngeal airway obturator comprising a body having an open central guiding channel, an intubation assembly comprising a video bougie or an optical stylet having a camera system at a distal end thereof, and an airway tube pre-loaded onto the video bougie or the optical stylet, where the intubation assembly is configured to be slidably seated within the central guiding channel of the oropharyngeal airway obturator, the obturator thereby providing a guided pathway for advancing the intubation assembly toward a patient's larynx.

In various embodiments, the central guiding channel includes a low-friction surface to facilitate sliding of the intubation assembly thereon.

In various embodiments, the obturator further includes a visual midline marker within the central guiding channel to provide a visual reference for centering the intubation assembly.

In various embodiments, the video bougie or the optical stylet includes a bent coude tip to facilitate navigation around anatomical structures including the tongue base and epiglottis.

In another aspect, a method for facilitating intubation of a remote patient is disclosed, which includes providing a video illustrating various steps of an intubation procedure to a novice operator (i.e., a medical professional with no or limited experience in airway management, such as intubation) via a communication network, establishing a communication link between the novice operator and an expert operator (i.e., a medical professional with extensive experience in airway management), where the expert operator facilitates intubation of a patient by the novice operator as the novice operator performs the intubation using information in the shared video by instructing the novice operator and responding to any question posed by the novice operator. The described method of airway intubation using the video bougie/optical stylet assembled with an endotracheal tube, and combined with an airway obturator greatly facilitates the intubation procedure and enhances safety and efficiency, and minimizes injury, hence making it much simpler and easier for a novice operator to follow direction provided by an expert operator.

Further understanding of various aspects of the present teachings can be obtained by reference to the following detailed description in conjunction with the associated drawings, which are described briefly below.

### BRIEF DESCRIPTION OF THE DRAWINGS

The accompanying drawings, which are incorporated in and constitute a part of this specification, illustrate various embodiments of the present teachings and together with the description, serve to explain the principles of the present teachings.

FIG. 1A is a perspective view of an oropharyngeal airway obturator according to an embodiment of the present disclosure.

FIG. 1B is another perspective view of the obturator shown in FIG. 1A.

FIG. 1C is yet another perspective view of the obturator shown in FIGS. 1A and 1B.

FIG. 1D is another perspective view of FIGS. 1A-1C.

FIG. 2 is a side view of the oropharyngeal airway obturator shown in FIG. 1A.

FIG. 3 is a front view of the oropharyngeal airway obturator, sectioned at plane BB and illustrating its generally U-shaped structure and central recess.

FIG. 4A is a side view of an exemplary video bougie that can be used with the obturator.

FIG. 4B is a cross-sectional side view of the distal end of the video bougie shown in FIG. 4A, detailing the internal camera system.

FIG. 4C is a perspective view of the distal end of the video bougie illustrated in FIGS. 4A and 4B.

FIG. 4D is an exploded perspective view detailing the components of the camera system of the video bougie.

FIG. 5A is an exploded perspective view of another embodiment of a video bougie featuring a tip-mounted assembly with a plurality of light-emitting diodes (LEDs).

FIG. 5B is a perspective view of the tip assembly of the video bougie of FIG. 5A, detailing the openings for the LEDs.

FIG. 6A is a perspective view of another embodiment of a video bougie in which the functions of forward illumination for video visualization and side-emission for transillumination are separated.

FIG. 6B is a top-down view of the distal tip of the video bougie of FIG. 6A, showing dedicated windows for the side-mounted LEDs used for transillumination.

FIG. 7 is a schematic view of an intubation system illustrating the functional relationship between the oropharyngeal airway obturator and an endotracheal tube. The endotracheal tube can be a component of an intubation assembly that further includes a video bougie for guiding the endotracheal tube.

FIGS. 8A-8E are a sequence of schematic views illustrating a method of employing the oropharyngeal airway obturator for intubating a patient, where FIG. 8A shows an operator opening a patient's mouth prior to insertion, FIG. 8B shows the insertion of the obturator into the patient's oropharynx to retract the tongue. FIG. 8C shows the placement of an intubation assembly into the guiding recess of the obturator. FIG. 8D shows the advancement of the intubation assembly along the obturator towards the larynx under visual guidance. FIG. 8E shows the endotracheal tube correctly positioned in the trachea and the subsequent withdrawal of a video bougie or an optical stylet.

FIG. 9 shows an endoscopic image showing a clear view of a patient's posterior larynx, including the epiglottis and arytenoids, as captured by an optical stylet before intubation.

FIG. 10 shows an endoscopic image showing an unobstructed view inside a patient's trachea, illustrating the view after the endotracheal tube has passed through the vocal cords.

FIG. 11 is a line graph illustrating the changes in patient heart rate over time (from baseline to 5 minutes post-intubation) for three different intubation methods compared in a clinical study.

FIG. 12 is a line graph showing the changes in patient systolic blood pressure over time for the three study groups.

FIG. 13 is a line graph showing the changes in patient diastolic blood pressure over time for the three study groups.

FIG. 14 is a line graph showing the changes in patient oxygen saturation levels over time for the three study groups.

FIG. 15 is a graph visualizing the percentage change in heart rate from baseline following intubation for the three study groups.

FIG. 16 is a graph visualizing the percentage change in systolic blood pressure from baseline following intubation for the three study groups.

FIG. 17 is a graph visualizing the percentage change in diastolic blood pressure from baseline following intubation for the three study groups.

FIG. 18 schematically depicts the performance of an intubation procedure by a novice medical professional under the guidance of a medical professional with an extensive experience in performing intubations.

### DETAILED DESCRIPTION

The present disclosure relates generally to intubation systems and methods designed for safer and more efficient airway management. In various embodiments, an intubation system according to the present teachings includes three main components: an oropharyngeal airway obturator, a video bougie or an optical stylet, and an airway tube. In various embodiments, the video bougie or the optical stylet is inserted through the airway tube to form a single intubating assembly, where the video bougie or the stylet provides real-time video visualization to guide the placement of the tube. The obturator is configured to function as a separate, pre-placed anatomical guide that can readily and slidably couple with this intubation assembly, retracting the tongue and guiding the intubation assembly accurately into the patient's airway.

An important aspect of the intubation system's functionality lies in the unique interaction between the obturator and the video bougie-tube or the stylet-tube assembly. In various embodiments, the obturator features an integrated, anatomically curved guiding channel specifically shaped to receive and constrain the intubation assembly, creating a stable yet relatively flexible mechanical interface. In practice, after the obturator is inserted into a patient's mouth to depress and retract the tongue, the intubation assembly is placed into this guiding channel. The obturator then functions as a “rail” or a “conduit,” along with visual midline marker/line, guiding the tip of the video bougie or the optical stylet directly and centrally toward the laryngeal inlet. This synergy between mechanical guidance from the obturator and direct visualization from the video bougie or the optical stylet simplifies and expedites the performance of the intubation procedure, allowing the operator to precisely place the airway tube into the larynx quickly and accurately (generally in one attempt), while minimizing trauma or mucosal injury, thereby transforming the intubation process into an easier and fully guided, visualized and more reliable procedure. This allows more novice operators, who do not have the benefit of prior specialized training in airway management, to achieve successful intubation.

The term “bougie,” as used herein, refers to an instrument having an intraluminal segment that can be inserted into or through a body passageway (lumen), such as trachea, esophagus, or urethra, for visualization of the lumen, guidance of other instruments through the lumen, widening of strictures, or dislodgement of obstructions within the lumen, among other applications.

The term a “low-friction surface” as used herein refers to a surface region of the obturator that presents a low resistance to sliding contact with a video bougie or an optical stylet or an intubation system comprising an endotracheal tube loaded onto a video bougie or an optical stylet, such that the intubation system can be advanced and withdrawn along the surface with minimal drag under normal clinical manipulation. In certain embodiments, the low-friction surface is characterized by a coefficient of kinetic friction between the intubation system and the surface that is less than about 0.02. The low-friction surface may be provided by selection of a bulk material for forming the obturator and/or by a surface treatment or coating, including without limitation fluoropolymer coatings, hydrophilic or other lubricious coatings, parylene, silicone-based finishes, or ultra-high molecular weight polyethylene, or various categories of plastic by way of example.

The term “red light source,” as used herein, refers to a light source that can generate light at a wavelength in a range of about 600 nm to about 700 nm, and preferably at a wavelength in a range of about 660 nm to about 680 nm.

The term “about,” as used herein, indicates a variation of at most 10% around a numerical value.

The term “substantially,” as used herein, indicates that a variation from a complete state or condition of at most 10%.

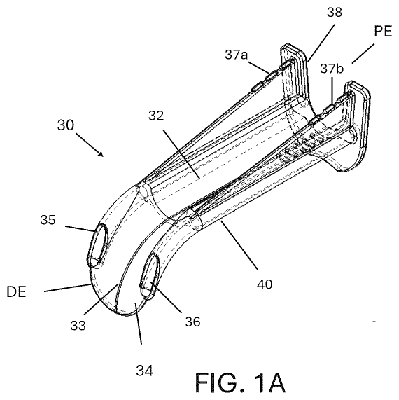

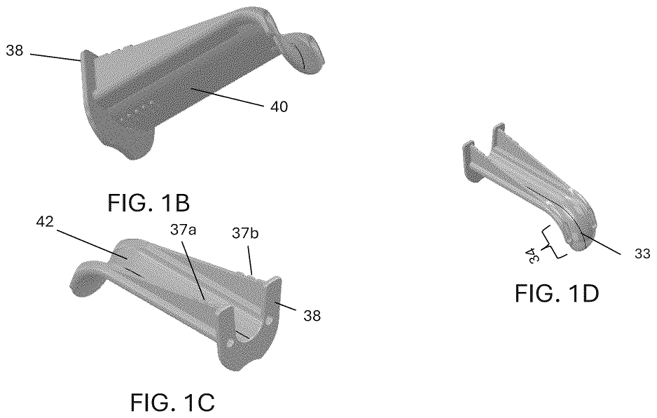

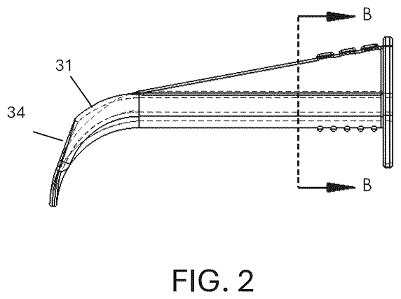

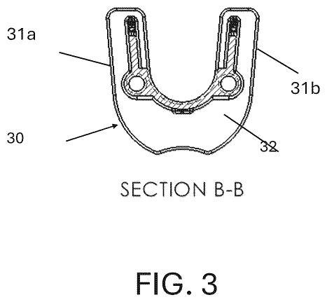

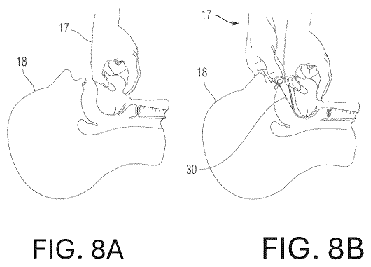

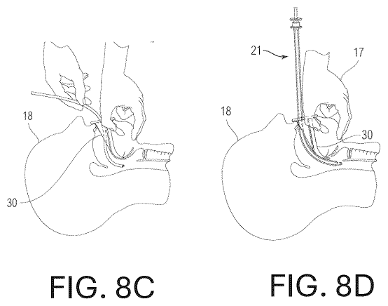

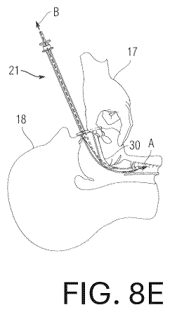

FIGS. 1A, 1B, 1C, 1D, 2 and 3 schematically illustrate an embodiment of an oropharyngeal airway obturator 30 of the present disclosure that extends from a proximal end PE to a distal end DE. As best seen in the sectioned view of FIG. 3, the obturator 30 has a generally U-shaped structure formed by a curved body 31. This U-shape is defined by two upwardly curving side portions 31a/31b that form a deep, open central recess 32 between them. In this embodiment, the obturator is designed to work in concert with a guiding instrument to facilitate safe and rapid placement of an airtube into the patient's larynx, such as a video bougie or an optical stylet 21 (see, FIG. 8D) coupled with an endotracheal tube. More specifically, the central recess 32 provides a guiding channel that allows the insertion of a video bougie or an optical stylet coupled with an endotracheal tube.

The obturator 30 is designed with several distinct surfaces, each with a specific function. The surface 40 is the glossal surface, which is anatomically curved to follow the contour of the tongue. Its primary function is to mechanically depress and retract the tongue, preventing it from obstructing the airway. As noted above, the obturator 30 further includes the guiding recess 32. In various embodiments, this recess is dimensioned to slidably receive an intubating assembly comprising of a video bougie or an optical stylet coupled to an endotracheal tube and maintaining its position along the patient's midline. By way of example, and without limitation, the guiding recess 32 can have a width in a range of about 5 mm to about 12 mm and a depth in a range of about 2 mm to about 6 mm. In some embodiments, the recess can have a tapered width along its length.

A midline black line 33 printed within this recess provides the operator with a clear visual cue to ensure the instrument remains centered. The proximal end of the device features bite-blocks 37a/37b and a faceplate 38 to provide a stable anchor and prevent over-insertion. A distal portion 34 of the obturator is shaped to rest in the hypopharynx above the epiglottis, completing the tongue retraction.

In this embodiment, obturator 30 is further equipped with passageways 35 and 36 (herein also referred to as conduits) that extend from the proximal end to the distal end of the obturator to provide ancillary functions during the intubation procedure. For example, one of the passageways 35/36 can be used as a conduit for connection of a suction catheter for applying suction to clear the patient's airway of secretions, saliva, and/or blood. The other passageway can be used as a conduit for delivering a flow of oxygen or inhalant gases directly to the patient's pharynx, helping to maintain oxygenation throughout the process. These integrated conduits enhance the device's utility by allowing simultaneous suction and oxygenation without interfering with the primary task of intubation.

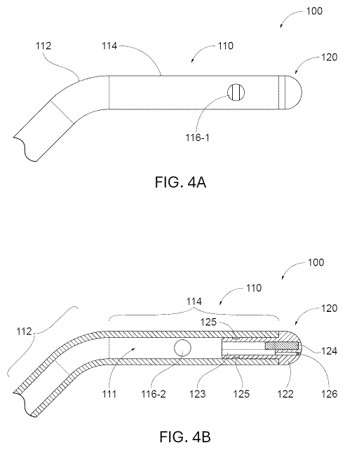

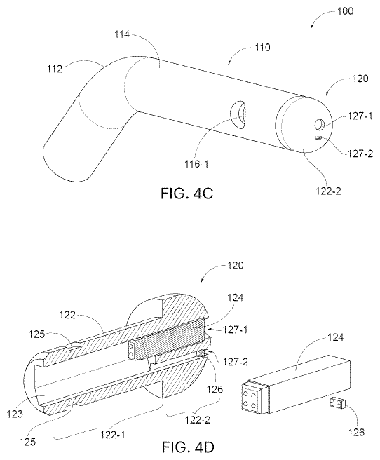

In various embodiments, the guiding instrument can be a video bougie that provides direct, real-time visualization of the anatomical lumen during intubation, thereby transforming the intubation process from a blind or semi-blind procedure into a fully guided one. An exemplary embodiment of such a video bougie 100 is illustrated in FIGS. 4A-4D. As shown in FIG. 4A, the distal end section of a video bougie 100 comprises an elongated, semi-rigid body part 110 with a proximal bent (and/or bendable) “coude” tip 112 to facilitate navigation around the base of the tongue and epiglottis, and a straight distal section 114 that houses the imaging components. The core of this device is the integrated camera system 120 embedded at the distal end.

The construction and assembly of a video bougie suitable for use in the practice of various embodiments of the present teachings are detailed in the cross-sectional views. As shown in FIG. 4B, the body part 110 is formed with a hollow interior channel 111 that serves as a conduit and housing for the wiring and hardware of the camera system 120. The camera system itself, detailed in FIG. 4D, includes a housing 122 containing a miniature video camera 124 and an adjacent light source 126, such as a white LED. The housing features an atraumatic, hemispherical head section 122-2 with a camera window 127-1 and a lighting window 127-2 to protect the sensitive components while allowing for clear imaging and illumination. An important aspect of the assembly involves securing the camera system within the bougie. The housing's arm section 122-1 includes a peripheral groove 125 that aligns with a hole 116-1 in the bougie's body. By way of example, during manufacturing, epoxy can be injected through this hole to fill the groove, thereby mechanically locking the camera system firmly in place and providing robust strain relief for the internal wiring.

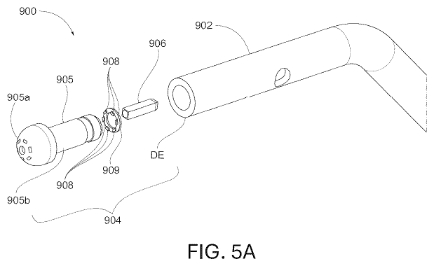

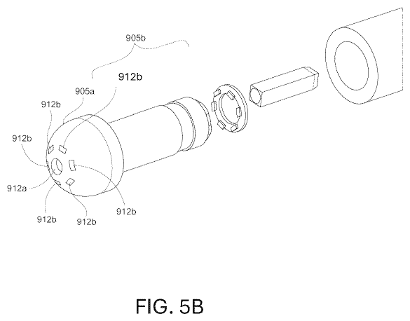

FIGS. 5A and 5B schematically depict a video bougie 900 according to another embodiment that includes a tip-mounted assembly 904 that integrates a plurality of light emitting diodes (LEDs) 908 around the central video camera 906. By way of example, these LEDs 908 can be a mix of white LEDs, which provide primary illumination for the video camera via openings 912b, and red LEDs. By way of example, the red light generated by the red LEDs can be specifically chosen for its ability to penetrate soft tissue. For example, in various embodiments, the red light can have a wavelength in the range of about 650-680 nm. In various embodiments, when the red light is activated, it can be seen externally through the patient's neck, allowing the operator to visually confirm that the bougie is in the trachea (where the light may highlight the tracheal rings) and not mistakenly placed in the esophagus.

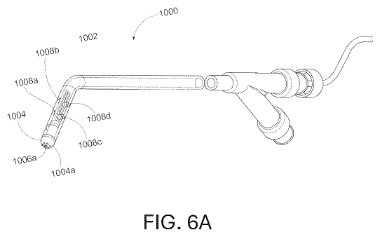

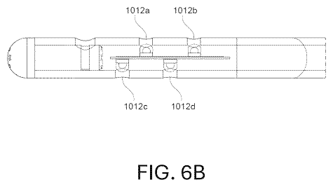

By way of further illustration, FIG. 6A shows an embodiment of the video bougie 1000 in which the functions of forward visualization and transillumination are separated for improved performance. More specifically, the video bougie 1000 device has a standard tip-mounted camera assembly 1004 viewing through opening 1006a with its own white light source 1004a for navigating the airway. In addition, a series of brighter, side-mounted red LEDs 1008a, 1008b, 1008c, and 1008d are positioned within the bent “coude tip” section of the body part 1002. These red LEDs are mounted on an internal circuit board 1010 and are designed to face outward, emitting a powerful red light through dedicated windows 1012a-d, shown in FIG. 6B, in the bougie's wall. This side-emission design provides a much brighter and more easily detectable external glow, offering unambiguous, real-time feedback on the bougie's location and trajectory within the neck, thereby maximizing the safety and accuracy of the intubation.

It should be understood that a guiding instrument that may be employed with an obturator to form an intubation system according to the present teachings is not limited to the video bougie discussed above.

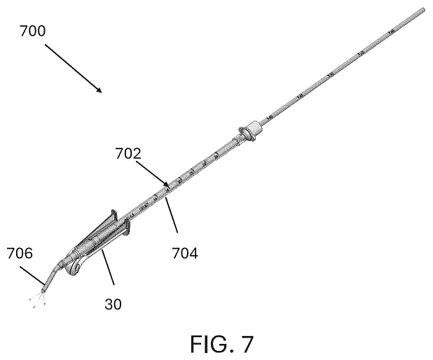

FIG. 7 schematically depicts an intubation system 700 which illustrates the functional relationship between the oropharyngeal airway obturator 30 and a separate intubation assembly 702. The obturator 30 serves as a pre-placed anatomical guide, while the intubation assembly 702 includes an endotracheal tube 704 which would have been pre-loaded onto a video bougie or an optical stylet 706, such as the video bougies described previously. The two components are coupled to create a stable and guided pathway for intubation.

The coupling is facilitated by a guiding interface provided by the geometry of the obturator 30. In particular, as discussed above with reference to FIGS. 1A-1D, 2, and 3, the obturator features a deep, U-shaped body 31 with a centrally located recess or groove 32 running along its pharyngeal surface 42. FIG. 7 shows the intubation assembly 702 seated snugly within this central groove 32 of obturator 30. In this embodiment, the width and the inner curvature of the obturator's groove are selected to substantially match the outer diameter and the curvature of the endotracheal tube 704. In various embodiments, the obturator can be provided in various color-coded sizes to match various sizes of the endotracheal tube ranging from the tube sizes used for neo-natal babies to those used for children and also adult male and female patients.

In various embodiments, this mechanical interface can be central to the method of use of an intubation system according to the present teachings. As discussed in more detail below, once the obturator 30 is inserted into the patient's mouth to retract the tongue, the intubation assembly 702 is then placed into this pre-established channel. The groove 32 constrains the assembly, preventing lateral deviation and automatically aligning it with the patient's anatomical midline. This allows the operator to advance the intubation assembly 702 with a simple, forward sliding motion along a stable, low-friction midline track. The obturator effectively acts as a “rail” or a “conduit,” guiding the tip of the video bougie (not shown in this figure) directly towards the laryngeal inlet. This guided coupling simplifies the intubation procedure, allowing the operator to focus on the video feed from the bougie to achieve precise placement of the endotracheal tube into the patient's larynx.

The oropharyngeal airway obturator can be manufactured from a variety of medical-grade polymeric materials, such as silicone, polyurethane, polypropylene or polyethylene, or plastic. In various embodiments, a lubricious material for the obturator is specifically chosen, or a lubricious coating is applied, to the surface of the central groove 32, to create a low-friction surface. In various embodiments, this characteristic can permit the intubation assembly, comprising the endotracheal tube and a video bougie or an optical stylet, to smoothly slide along the guiding channel with minimal resistance, facilitating an effortless and precise advancement into the patient's larynx. Soft and flexible rounded edges may be incorporated into the obturator to minimize trauma during insertion while a more rigid material may be used for the core portion of the obturator to achieve a stiffness desired for retracting the tongue. By way of example, such an obturator can be manufactured using an overmolding technique with two materials with different degrees of stiffness. Further, in various embodiments, the obturator is a single-use, disposable device that can be discarded after use with a patient.

Similarly, the components of the intubation assembly are formed from specific materials to ensure proper function. By way of example, the body of the video bougie itself may be formed from a range of polymeric materials including, without limitation, polyurethane, polyester, polyether block amide, polyvinyl chloride, and various low and high density polyethylene blends. In various embodiments, the bougie can be constructed with an inner polyester core and an outer resin coating to achieve a desired balance of flexibility and durability, while the endotracheal tube is typically made from a clear and flexible material like medical-grade, transparent polyvinyl chloride (PVC) to conform to the patient's airway.

An example of a method of employing the obturator 30 is illustrated schematically in the sequence of drawings presented in FIGS. 8A-8E. The process begins with a patient 18 in an initial resting position. An operator 17 lifts the patient's mandible or jaw, typically with the non-dominant hand, to open the oral cavity. Following this, the oropharyngeal airway obturator 30 is inserted into the patient's mouth and advanced into the oropharynx. Once seated, the obturator's curved glossal surface depresses and retracts the tongue, while its distal end rests in the hypopharynx, thereby creating and maintaining a patient upper airway pathway.

With the obturator 30 in place, the intubation proceeds as follows. An intubating assembly 21, which includes a video bougie or an optical stylet preloaded with an endotracheal tube, is introduced into the patient's mouth with the operator's dominant hand. As depicted in FIG. 8C, the tip of the assembly 21 is placed into the midline recess 32 on the pharyngeal surface of the obturator. A black reference line on the midline of the obturator provides a visual cue to help the operator follow the midline path during the insertion of the video bougie or the optical stylet.

The obturator 30 can now function as a guide. The operator advances the intubating assembly 21 along the channel provided by the obturator's recess. The midline black line 33 on the obturator's surface provides a continuous visual reference to ensure the assembly remains centered as it moves towards the larynx while the video bougie or optical stylet allows for continuous visualization of the airway throughout this advancement. The operator can see anatomical landmarks such as the uvula, the base of tongue, the vallecular, the epiglottis and vocal cords in real-time.

The operator continues to advance the assembly 21 under direct visualization, navigating it through the vocal cords and into the trachea. FIG. 8E shows the endotracheal tube (A) correctly positioned within the trachea, having been successfully guided past the obturator 30. Subsequently, the video bougie or the optical style (B) is withdrawn from the endotracheal tube, leaving the endotracheal tube in place securely in the airway. The obturator is then carefully removed from the patient's mouth.

An intubation system according to various embodiments including a combination of an obturator and a video bougie provides distinct advantages over conventional intubation systems and methods of their use. By way of example, and without limitation, the combination of the video bougie and the obturator can eliminate the need for a bladed laryngoscope and reduce potential trauma to a patient's lips and teeth. Further, it simplifies and expedites the intubation process. For example, often a second set of hands is needed to hold a laryngoscope during an intubation procedure. In contrast a single operator can perform an intubation procedure using an intubation system, including a video bougie or an optical stylet, according to various embodiments of the present teachings. In particular, a video generated from the tip of a video bougie can provide a better view of the intubation procedure than a laryngoscope because it moves into the larynx and is not held in a fixed position. Furthermore, in various embodiments, a video bougie can provide a wider-angle image than one that can be obtained via an optical stylet, even if the stylet has a video camera on its proximal end, due to narrow lens optics. If unequipped with a camera, looking into an optical stylet's eyepiece limits the view of the rest of the patient's anatomical structures, unlike an image displayed on a monitor.

Another distinction is that in various embodiments, unlike most bladed laryngoscopes and optical stylets, an intubation system including a video bougie and an obturator can be configured to be a single-use disposable tool. This feature can prevent cross contamination between patients and eliminates the need (and the cost) for reprocessing and cleaning instruments.

Further, unlike a Berman Oral Airway or a Guedel airway, an obturator according to various embodiments provides a groove that is designed to accommodate an endotracheal tube (ETT). While an ETT typically features an inflatable cuff that can be used to prevent aspirating fluids (e.g., saliva and/or blood) into a patient's lungs, the Berman or Guedel type oral airways do not prevent the patient from aspirating fluids. In contrast, the Shikani obturator, such as those described above, can be connected to continuous suction throughout the intubation procedure, hence removing saliva/secretion or blood that could otherwise enter the airway. Moreover, as discussed above, in various embodiments, an obturator according to the present teachings can include integrated passageways configured for application of suction and delivery of oxygen to a patient. Neither the Berman or Guedel type oral airways does feature such integrated passageways; nor do they have protruding bosses provided on the bite block of various embodiments of an obturator according to the present teachings, which engage a patient's teeth to prevent accidental dislodgement.

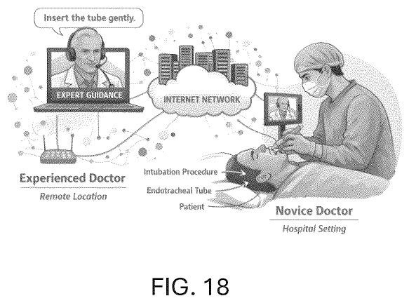

In another aspect, the present disclosure provides a method for facilitating the performance of an intubation of a remote patient. For example, with reference to FIG. 18, in one embodiment, a medical professional, such as a doctor, who is experienced in intubating patients can be connected, e.g., via a communication network, such as the Internet, to a less experienced (such as a novice) medical professional who will perform the intubation. The experienced medical professional can see in a live and continuous fashion, the view as seen by the tip of the video-bougie/optical stylet and guide the less experienced professional throughout the intubation procedure as well as respond to questions that the less experienced professional may have.

In various embodiments, an instruction video providing step-by-step instructions regarding how to perform the intubation, e.g., instructions for performing intubation methods according to the present teachings, can be shared with the less experienced professional, e.g., by a third party, so that the less experienced professional can view the procedure prior to performing it under the guidance of the more experienced professional. This would provide a valuable teaching tool for novice operators who can hence learn how to intubate safely and efficiently.

### Clinical Study and Results

To validate the efficacy and advantages of using an optical stylet designed by Dr. Alan Shikani (which is herein referred to as Shikani Optical Stylet (SOS)) for intubation, a research study was conducted. The SOS included a malleable J-shaped stylet-scope with polymeric light-carrying fibers that was coupled to an endotracheal tube as a single intubating unit and allowed continuous visualization of a patient's airway while intubating and placing the tube at a proper distance into the trachea. The SOS can be cambered to the proper airway curvature, can be maneuvered around a floppy epiglottis to provide a clear view of the vocal cords and can be advanced through the glottis into the trachea.

The study compared the use of the SOS together with a tongue-retracting device against traditional blade laryngoscopy. The results demonstrated that employing an optical stylet, particularly when combined with a tongue-retracting device-which serves as a functional analog for the obturator of the present teachings-provides significant improvements in patient safety and hemodynamic stability.

The research study involved 60 patients randomly divided into three groups of 20. For intubation of patients in Group 1, only the video optical stylet was used. For intubation of patients in Group 2, a combined approach using both the video optical stylet and a Macintosh blade was employed; this group serves as a clinical proxy for the intubation system of the present teachings, where the blade performs the tongue-retraction function of the obturator. Patients in Group 3 were intubated using the Macintosh blade alone.

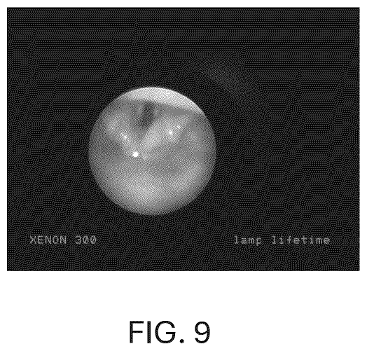

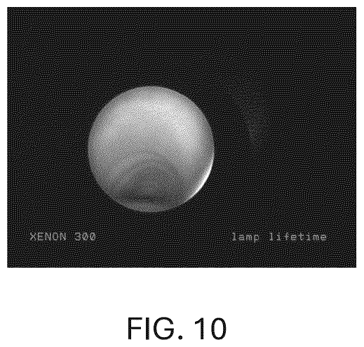

The superior visualization afforded by the optical stylet is highlighted in endoscopic images from the study. As shown in FIG. 9, the optical stylet provided a clear, direct view of the posterior larynx, including the epiglottis and arytenoids, which is critical for accurate tube placement. The image presented in FIG. 10 further shows an unobstructed view inside the trachea after the endotracheal tube was passed through the vocal cords. While all patients were successfully intubated, the data presented in Tables 1 and 2 below reveals important differences in outcomes. Complications such as lip laceration, mucosal injury, and hoarseness were only observed in the groups in which a blade was used (one minor trauma in Group 2 and three in Group 3). Notably, there were zero complications recorded in Group 1, where only the less-traumatic optical stylet was used.

TABLE 1

| | | Weight | Height | Malampati |
|---|---|---|---|---|
| | Sex | (mean) | (mean) | Classification |
| Group 1 | 14 M, 7 F | 174.15 | 5′ 46 | 1.70 |
| (Video-SOS) | | | | |
| Group 2 | 8 M, 14 F | 167.95 | 5′ 37 | 1.75 |
| (Video-SOS + | | | | |
| Blade) | | | | |
| Group 3 | M, 14 F | 167.95 | 5′ 45 | 1.80 |
| (Blade) | | | | |

TABLE 2

| | Dental | Lip | Mucosal | Hoarse- |
|---|---|---|---|---|
| | Injury | Injury | Injury | ness |
| Group 1 | 0 | 0 | 0 | 0 |
| (OSL) | | | | |
| Group 2 | 0 | 0 | 0 | 0 |
| (Video-SOS + | | | | |
| Blade) | | | | |
| Group 3 | 0 | 1 | 1 | 1 |
| (Blade) | | | | |

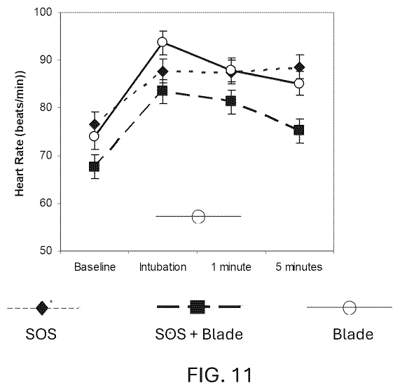

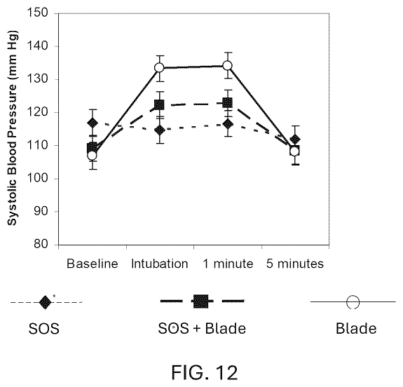

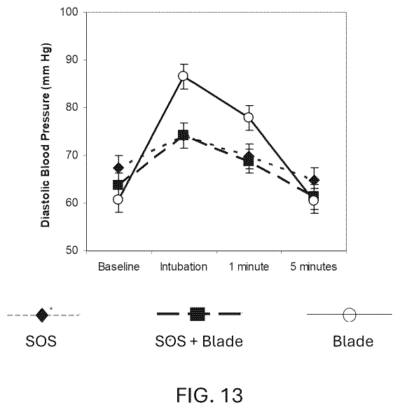

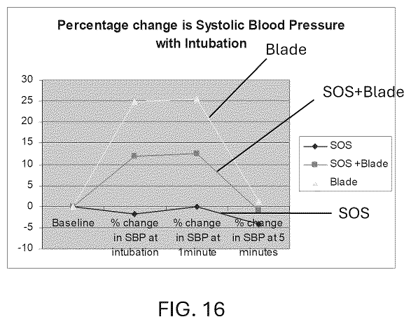

The study also monitored hemodynamic responses to intubation. FIG. 11 shows the change in heart rate following intubation, which increased across all groups. In contrast, FIG. 12 and FIG. 13 illustrate a significantly more stable cardiovascular response in patients intubated with the optical stylet. As shown in FIG. 12 and detailed in the percentage-change visualization of FIG. 16, the blade-only group experienced a sharp systolic blood pressure increase of 25%, a response significantly higher than that of the optical stylet-only group.

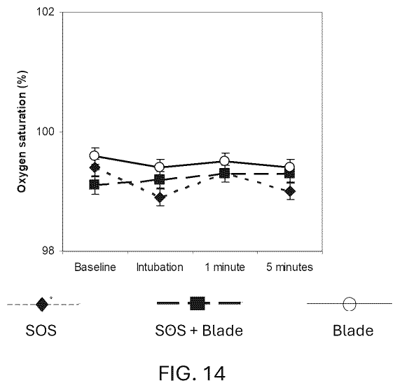

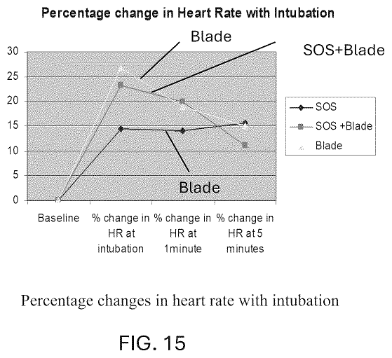

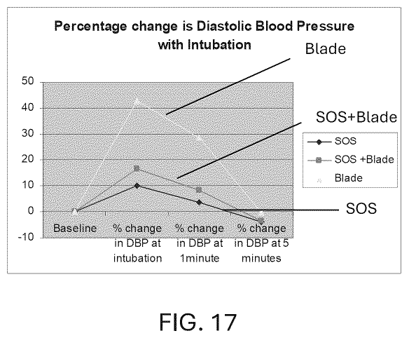

This trend is even more pronounced in the diastolic blood pressure readings shown in FIG. 13 and the corresponding percentage-change graph in FIG. 17. At the moment of intubation, the blade-only group demonstrated a dramatic 43% increase in diastolic pressure, which was significantly higher than both the optical stylet-only and the combined-approach groups. This reduced pressure response indicates that the optical stylet intubation technique was less stimulating and traumatic for the patient. Finally, as shown in FIG. 14, there were no significant changes in oxygen saturation levels among the groups, confirming that all methods were effective at maintaining patient oxygenation. In summary, the clinical data strongly supports the conclusion that intubation using the optical stylet, as facilitated by a tongue-retracting device like the obturator, leads to fewer physical complications and a significantly attenuated hemodynamic response compared to traditional blade laryngoscopy.

Those having ordinary skill in the art will appreciate that various changes can be made to the above embodiments without departing from the scope of the present teachings.

What is claimed is:

1. A kit for facilitating airway intubation, comprising:
a video bougie comprising an elongated body and a camera system disposed at a distal end thereof, the camera system configured to generate video images of an anatomical lumen, and
an oropharyngeal airway obturator comprising a body configured to slidably receive said video bougie to facilitate insertion of an endotracheal tube into a patient.

2. The kit of claim 1, wherein said obturator comprises a guiding channel extending along at least a portion of its length and dimensioned to slidably receive the video bougie.

3. The kit of claim 1, wherein said obturator comprises a generally U-shaped body defined by two upwardly curving side portions between which said guiding channel is formed.

4. The kit of claim 3, wherein said guiding channel is formed along a central portion of said generally U-shaped body.

5. The kit of claim 1, wherein the obturator further comprises at least one proximate bite-block configured to prevent dislodgement of the obturator from a patient's mouth and a proximal flange configured to prevent over-insertion of the obturator into a patient's mouth.

6. The kit of claim 1, wherein the obturator body further comprises at least one ancillary conduit configured for at least one of application of a suction or delivery of oxygen to the patient.

7. The kit of claim 6, wherein said at least one ancillary conduit comprises a pair of conduits, wherein one of said conduits is configured to the application of a suction and the other conduit is configured for delivery of the oxygen.

8. The kit of claim 2, wherein the obturator body further comprises a reference marker printed along at least a portion of a central axis thereof to provide a visual cue to an operator for facilitating insertion of an endotracheal air tube using said video bougie.

9. The kit of claim 1, wherein the video bougie further comprises a red light source configured to provide transillumination through a patient's tissue.

10. The kit of claim 9, wherein the at least one red light source comprises a plurality of side-mounted light emitting diodes (LEDs) configured to emit light through one or more windows in a wall of the video bougie.

11. The kit of claim 10, further comprising an endotracheal tube (ETT) configured to be pre-loaded onto the video bougie to form an intubation assembly.

12. The kit of claim 1, wherein the obturator is formed of a polymeric material and wherein said guiding channel has a low-friction surface to facilitate sliding said video bougie along a surface of said guiding channel.

13. The kit of claim 1, wherein said guiding channel includes a groove.

14. An oropharyngeal airway obturator, comprising:
a body extending from a proximal end to a distal end, the body having an anatomically curved glossal surface and a pharyngeal surface; a guiding channel formed along the pharyngeal surface, the channel being open along its length and dimensioned to slidably receive an intubating assembly; and
at least one bite-block and a flange disposed at the proximal end.

15. The oropharyngeal airway obturator of claim 14, wherein said guiding channel is a central guiding channel.

16. The obturator of claim 15, wherein the body has a generally U-shaped cross-section defining the central guiding channel.

17. The obturator of claim 16, further comprising a visual midline marker printed on a top surface of the central guiding channel.

18. The obturator of claim 12, wherein the body further comprises at least two ancillary conduits extending from the proximal end toward the distal end, a first conduit for suction and a second conduit for oxygen delivery.

19. The obturator of claim 12, wherein said body is formed of a polymeric material and said guiding channel has a low-friction surface.

20. A method for intubating a patient, the method comprising the steps of:
inserting an oropharyngeal airway obturator into a patient's mouth to retract the patient's tongue, the obturator having an open guiding channel,
placing an intubation assembly into the guiding channel of the inserted obturator, the intubation assembly comprising a video bougie or an optical stylet and an endotracheal tube (ETT),
advancing the intubation assembly along the guiding channel toward the patient's larynx while viewing real-time video images generated by a camera on the video bougie or by the optical stylet; and
guiding the airway tube into the patient's trachea under visual guidance from the video images.

21. The method of claim 20, further withdrawing the video bougie or the optical stylet from the ETT, and removing the oropharyngeal airway obturator from the patient's mouth.

22. The method of claim 20, further comprising the step of activating a red light source on the video bougie or the optical stylet to generate transillumination, and externally observing the transillumination to confirm a position of the video bougie or the optical stylet to facilitate said step of guiding the airway tube into the patient's trachea.

23. The method of claim 20, further comprising the step of administering suction or oxygen through at least one ancillary conduit in the oropharyngeal airway obturator during the intubation.

24. An intubation system, comprising:
an oropharyngeal airway obturator comprising a body having an open central guiding channel;
an intubation assembly comprising a video bougie or an optical stylet having a camera system at a distal end thereof, and
an airway tube pre-loaded onto the video bougie or the optical stylet;
wherein the intubation assembly is configured to be slidably seated within the central guiding channel of the oropharyngeal airway obturator, the obturator thereby providing a guided pathway for advancing the intubation assembly toward a patient's larynx.

25. The intubation system of claim 24, wherein the central guiding channel comprises a low-friction surface to facilitate sliding of the intubation assembly thereon.

26. The intubation system of claim 24, wherein the obturator further comprises a visual midline marker within the central guiding channel to provide a visual reference for centering the intubation assembly.

27. The intubation system of claim 24, wherein the video bougie or the optical stylet comprises a bent coude tip to facilitate navigation around anatomical structures.

28. A method for facilitating intubation of a remote patient, the method comprising:
sharing a video illustrating various steps of an intubation procedure with a novice operator via a communication network,
establishing a communication link between a novice operator and an expert operator having expertise in airway management,
wherein said expert operator facilitates intubation of a patient by the novice operator as the novice operator performs the intubation using information in the shared video by instructing the novice operator and responding to any question posed by the novice operator.
